(19) United States
(12) Patent Application Publication
(10) Pub. No.: US 2009/0054442 A1
(43) Pub. Date: Feb. 26, 2009

(54) METHOD OF ADMINISTERING OPHTHALMIC FLUIDS

(76) Inventors: BEN Z. COHEN, New York, NY (US); NIGEL KELLY, Rye Brook, NY (US)

Correspondence Address: HOFFMANN & BARON, LLP, 6900 JERICHO TURNPIKE, SYOSSET, NY, 11791, US

(21) Appl. No.: 12/259,752

(22) Filed: Oct. 28, 2008

Related U.S. Application Data
(63) Continuation of application No. 10/467,140, filed on Jan. 28, 2004, now abandoned.

(51) Int. Cl. A61K 31/498; A61K 31/215; A61K 31/433; A61P 27/02; A61K 31/382

(52) U.S. Cl. 514/249; 514/530; 514/362; 514/432

(57) ABSTRACT

A method is provided of treating an eye with ophthalmologically active compounds, particularly with intra-ocular pressure lowering (IOP) compounds, where a dose of 5-15 microliters is delivered at a standard concentration.

This application is a continuation of U.S. application Ser. No. 10/467,140, filed Jan. 28, 2004, now pending, which claims priority of U.S. Provisional Application No. 60/266,276, filed Feb. 2, 2001, and U.S. Provisional Patent Application No. 60/278,723, filed Mar. 26, 2001.

### BACKGROUND OF THE INVENTION

This invention relates to methods for administering ophthalmic agents and, more particularly, to methods of administering ophthalmic fluids.

In the prior art, it is well recognized that a large percentage of ophthalmic fluid medicine that is administered topically, particularly with a dropper bottle, is lost by drainage, either externally or through nasolacrimal drainage. Techniques have been developed to minimize this waste, such as with the typical practice of punctal occlusion, where a patient places a finger over a corner of their eye to physically block drainage for a period of time after applying a dose of fluid medicant. P.36, Biopharmaceutics of Ocular Drug Delivery, Peter Edman, 1993. Others have altogether taken a different approach suggesting oral administration of ophthalmologically active compounds, such as in U.S. Pat. No. 6,297,240 to Embleton, issued Oct. 2 2001.

Generally, recommended dose volumes of ophthalmic fluid medication are in the range of 30-70 microliters for intra-ocular pressure lowering (IOP) compounds used in treating glaucoma. It has been found that a reduction in dose volume down to 20 microliters leads to an increase in efficacy of the medication. Miller, K., Brown, R. H., Lynch, M. G., Eto, C. Y., Lue, J. C., and Novak, G. D., Does Drop Size Influence The Efficacy Of A Topical Beta Blocker?, Invest. Ophthalmol. Vis. Sci., Suppl. 27, 161, 1986. Although the prior art has contemplated the use of doses as small as 10 microliters, the literature has indicated that the concentration of IOP compounds in such small doses should be increased to maintain efficacy. Miller, et at.

### SUMMARY OF THE INVENTION

With the subject invention, a method of treating an eye is provided with ophthalmologically active compounds, particularly with intra-ocular pressure lowering (IOP) compounds, where a dose of 5-15 microliters is delivered at a standard concentration. It has been found by the inventors herein that contrary to conventional wisdom, the concentration of an ophthalmologically active agent in a fluid medicant does not need to be increased to maintain the efficacy of the drug. Rather, it has been found that because a smaller dose of fluid medication causes less irritation of the eye than with larger doses, reflex tearing and reflex blinking are minimized. The concentration of the medication in the tear film is higher than with larger doses, due to the dispersion of the drug in a smaller liquid volume defined by the tear film. Also, advantageously, a greater percentage of the medication is absorbed into the eye and not absorbed systematically after draining. With 5-15 microliter doses, the therapeutic effects of the drug are at least as great as with larger doses, even though administered in a smaller amount. Typical standard doses that are administered (30-70 microliters) cause reflex blinking and reflex tear generation which, in turn, cause a fairly rapid drainage of the dose, both externally and through the nasolacrimal duct. A dose of 5-15 microliters causes minimal reflex blinking and-reflex tear generation, resulting in slower drainage than with larger doses.

The inventive method can be used with IOP agents, and other classes of ophthalmic fluids, such as antibiotics, diagnostic agents, anti-inflammatory agents, and artificial tears and eye whiteners.

As used herein, “standard concentration” shall refer to an ophthalmologically active compound's concentration in a dose of 30-70 microliters of ophthalmic fluid such as the IOP compound concentration in a dose of 30-70 microliters. For example, latanoprost sold by Pharmacia Corporation under the trademark “Xalatan” has a standard concentration of 50 micrograms of latanoprost per milliliter (0.005%) for a recommended dose of 30 microliters; thus, one 30 microliter dose (one drop) contains 1.5 micrograms of latanoprost. Timolol maleate sold by Merck & Co., Inc. under the trademark “TIMOPTIC” is prepared at two different standard concentrations. In one standard concentration (referred to as “TIMOPTIC 0.25%), each milliter of fluid contains 2.5 milligrams of timolol (3.4 milligrams of timolol maleate); while, with a second standard concentration (referred to as “TIMOPTIC 0.5%”), each milliliter of fluid contains 5.0 milligrams of timolol (6.8 milligrams of timolol maleate). Both “TIMOPTIC” fluid medications are administered in one drop doses of approximately 30 microliters. Dorzolamide hydrochloride sold under the trademark “TRUSOPT” by Merck & Co., Inc. has a standard concentration of 20 milligrams of dorzolamide (22.3 milligrams of dorzolamide hydrochloride) per millimeter (2%). Brimonidine tartrate sold under the trademark “ALPHAGAN” by Allergan, Inc. has a standard concentration of 2 milligrams of brimonidine tartrate (equivalent to 1.32 milligrams as brimonidine free base) per milliliter (0.2%); whereas, brimonidine tartrate sold under the trademark “ALPHAGAN P”, also sold by Allergan, Inc., has a standard concentration of 0.15%.

### DETAILED DESCRIPTION OF THE SUBJECT INVENTION

With the subject invention, a method of treating an eye is provided with ophthalmologically active compounds, particularly with IOP compounds, where a dose of 5-15 microliters is delivered at a standard concentration. In a preferred manner of practicing the invention, the dose is delivered to the eye without punctal occlusion.

Suitable ophthalmologically active compounds that may be used with the subject invention include

I. anti-glaucoma/IOP compounds such as:
a.) alpha-adrenoceptor blocking agents, e.g., apraclonidine, brimonidine, AGN 192836, AGN 193080, etc.
b.) beta-adrenoceptor blocking agents, e.g., carteolol, betaxolol, levobunolol, metipranolol, timolol, vaninolol, adaprolol, etc.
c.) miotics, e.g., pilocarpine, carbachol, physostigmine, etc.
d.) sympathomimetics, e.g., adrenaline, dipivefrine, etc.
e.) carbonic anhydrase inhibitors, e.g., acetazolamide, dorzolamide, etc.; and,
f.) prostaglandins, e.g., PGF-2 alpha or its prodrug latanoprost;

II. diagnostic fluids, such as anesthetics and medications to dilate the eye;

III. anti-inflammatory agents (both steroid and non-steroid);

IV. artificial tears and eye whiteners; and

V. antibiotics.

The inventive method is particularly well-suited to work with beta-adrenoceptor blocking agents (e.g., timolol maleate); prostaglandins (e.g., latanoprost); alpha-adrenoceptor blocking agents (e.g., brinonidine tartrate); and, carbonic anhydrase inhibitors (e.g., dorzolamide hydrochloride).

In administering the dose of 5-15 microliters, the concentration of the ophthalmologically active compound in the fluid medication need not be any greater than its standard concentration. Thus, with respect to prostaglandins (e.g., latanoprost), the concentration of the dose need not be any greater than 0.005%; with respect to beta-adrenoceptor blocking agents (e.g., timolol), no greater than 0.5%, and more preferably, no greater than 0.25%; with respect to carbonic anhydrase inhibitors (e.g., dorzolamide), no greater than 0.2%, and with respect to alpha-adrenoceptor blocking agents (e.g., brimonidine), no greater than 0.2%, and more preferably, no greater than 0.15%.

Tests were conducted to determine the efficacy of smaller doses having standard concentrations of ophthalmologically active compounds. In a first test, 15 microliter doses of TIMOPTIC 0.5% were administered to 20 subjects who did not have any history of suffering from glaucoma or elevated intra-ocular pressure. Standard concentration of the fluid was utilized. A 15 microliter drop was delivered into both eyes of each subject. A tonometer was used to measure ocular pressure before administration of the drug; two hours after administration of the drug; and six hours after administration of the drug. Table 1 sets forth the average ocular pressure drops at the two- and six-hour marks.

TABLE 1
15 Microliter Study

| | Avg. Ocular Pressure Drop- |
|---|---|
| Time Elapse (hrs) | 15 μl (%) |
| 2 | 30 |
| 6 | 26 |

In a second study, 10 microliter doses of TIMOPTIC 0.5% were administered to 24 subjects who did not have any history of suffering from glaucoma or elevated intra-ocular pressure. Standard concentration of the fluid was utilized. A 10 microliter drop was delivered into both eyes of each subject. A tonometer was used to measure ocular pressure before administration of the drug; two hours after administration of the drug; and six hours after administration of the drug. Table 2 sets forth the average ocular pressure drops at the two- and six-hour marks.

TABLE 2
10 Microliter Study

| | Avg. Ocular Pressure Drop- |
|---|---|
| Time Elapse (hrs) | 10 μl (%) |
| 2 | 28 |
| 6 | 26 |

With both tests, the results are equal to or exceed expected results of TIMOPTIC 0.5%.

Any device or method can be used to deliver the 5-15 microliter doses. Dispensers for delivering such doses are known in the prior art, such as U.S. Pat. No. 5,152,435, which issued Oct. 6, 1992; U.S. Pat. No. 5,881,956, which issued Mar. 16, 1999; and WIPO Published Patent Application No. WO 01/14245. The disclosures of these references are incorporated by reference herein in their respective entireties.

Various changes and modifications can be made to the present invention. It is intended that all such changes and modifications come within the scope of the invention as set forth in the following claims.

What is claimed is:

1. A method of treating an eye with ophthalmologically active compounds, said method comprising delivering a 5-15 microliter dose of fluid to the eye, said dose including at least one of the ophthalmologically active compounds at a standard concentration.

2. A method as in claim 1, wherein said delivering is performed without punctal occlusion.

3. A method as in claim 1, wherein said at least one ophthalmologically active compound is an intra-ocular pressure lowering compound.

4. A method as in claim 3, wherein said intra-ocular pressure lowering compound is selected from the group consisting of alpha-adrenoceptor blocking agents; beta-adrenoceptor blocking agents; miotics; sympathomimetics; carbonic anhydrase inhibitors; and prostaglandins.

5. A method as in claim 1, wherein said at least one ophthalmologically active compound is selected from the group consisting of antibiotics, diagnostic fluids, anti-inflammatory agents, artificial tears, and eye whiteners.

6. A method of treating an eye with prostaglandin, said method comprising delivering a 5-15 microliter dose of fluid to the eye, said dose having a concentration of prostaglandin no greater than 0.005%.

7. A method as in claim 6, wherein said prostaglandin is latanoprost.

8. A method of treating an eye with beta-adrenoceptor blocking agent, said method comprising delivering a dose of 5-15 microliters of fluid to the eye, said dose having a concentration of beta-adrenoceptor blocking agent no greater than 0.5%.

9. A method as in claim 8, wherein said beta-adrenoceptor blocking agent is timolol.

10. A method as in claim 9, wherein said concentration of said dose is no greater than 0.25%.

11. A method of treating an eye with carbonic anhydrase inhibitor, said method comprising delivering a dose of 5-15 microliters of fluid to the eye, said dose having a concentration of carbonic anhydrase inhibitor no greater than 0.2%.

12. A method as in claim 11, wherein said carbonic anhydrase inhibitor is dorzolamide.

13. A method of treating an eye with alpha-adrenoceptor blocking agent, said method comprising delivering a dose of 5-15 microliters of fluid to the eye, said dose having a concentration of alpha-adrenoceptor blocking agent no greater than 0.2%.

14. A method as in claim 13, wherein said alpha-adrenoceptor blocking agent is brimonidine.

15. A method as in claim 14 wherein said concentration of said dose is no greater than 0.15%.
